(19) United States
(12) Patent Application Publication
(10) Pub. No.: US 2022/0125541 A1
(43) Pub. Date: Apr. 28, 2022

(54) ALIGNMENT MECHANISM FOR CIRCULAR STAPLING INSTRUMENTS

(71) Applicant: Covidien LP, Mansfield, MA (US)

(72) Inventors: Adi TSALACH-WEISS, New Haven, CT (US); Drew R. SEILS, Guilford, CT (US); Steven H. JOYCE, Durham, CT (US); Stephen R. PAUL, Burlington, CT (US); Kelly A. AZEREDO, Killingworth, CT (US)

(21) Appl. No.: 17/498,807

(22) Filed: Oct. 12, 2021

Related U.S. Application Data
(60) Provisional application No. 63/105,528, filed on Oct. 26, 2020.

(51) Int. Cl. A61B 34/00; A61B 17/115; A61B 90/92

(52) U.S. Cl. CPC A61B 34/73; A61B 2017/00876; A61B 90/92; A61B 17/1155

(57) ABSTRACT

An adapter for a circular stapling instrument includes an elongate body supporting a loading unit and from which a trocar member extends. The trocar member includes a first magnetic member and an anvil assembly of the circular stapling instrument includes a second magnetic member. The second magnetic member is magnetically attracted to the first magnetic member to aid alignment of a center rod of the anvil assembly with the trocar member and facilitate attaching the anvil assembly to the trocar member

### CROSS-REFERENCE TO RELATED APPLICATION

This application claims the benefit of and prior to U.S. Provisional Patent Application No. 63/105,528, filed on Oct. 26, 2020, the content of which is incorporated herein by reference in its entirety.

### FIELD

The disclosure relates to circular stapling instruments. More particularly, the disclosure relates to mechanisms for facilitating the attachment of an anvil assembly circular stapling instrument to a trocar member of the circular stapling instrument.

### BACKGROUND

Circular stapling instruments are used to perform end to end anastomosis. During a typical surgical stapling procedure, an anvil assembly of the circular stapling instrument is delivered to the surgical site while a staple cartridge of the circular stapling instrument supported on an elongate shaft is inserted through an incision in, for example, the abdominal wall. Alternatively, the circular stapling instrument may include a curved or flexible shaft that permits introduction of the staple cartridge to the surgical site through a natural body orifice, e.g., mouth or anus. The circular staplers may be manual or powered, and may be modified for use with robotic surgical systems.

To better facilitate attachment of the anvil assembly to a trocar member extending from the elongate body and through the staple cartridge of the circular stapling instrument, it would be beneficial to have a mechanism for attracting the anvil assembly and the trocar member towards one another.

### SUMMARY

A circular stapling instrument including an elongate body having a distal portion, a loading unit disposed on the distal portion of the elongate body, a trocar member extending from the elongate body and through the loading unit, and an anvil assembly. The loading unit includes a staple cartridge supporting a plurality of surgical staples. The trocar member includes a first magnetic member supported along a length of the trocar member. The anvil assembly includes a center rod having a plurality of legs defining an opening. The center rod includes a second magnetic member supported on the plurality of legs of the center rod. The second magnetic member is magnetically attracted to the first magnetic member to direct the trocar member towards the anvil assembly.

In certain aspects of the disclosure, the trocar member includes a piercing distal tip and a notch. The first magnetic member may be disposed between the piercing distal tip and the notch. The trocar member may include a visible band. The visible band may be disposed within the notch. The center rod of the anvil assembly may be releasably engageable with the trocar member. The visible band may be covered by the plurality of legs of the center rod when the center rod is attached to the trocar member. The visible band may be orange. A distal tip of the trocar member may be configured to pierce tissue.

In some aspects of the disclosure, the first magnetic member may include a magnetic ring. In other aspects of the disclosure, the first magnetic member includes a magnetic sleeve. The elongate body may be a component of an adapter assembly.

### BRIEF DESCRIPTION OF THE DRAWINGS

The accompanying drawings, which are incorporated in and constitute a part of this specification, illustrate aspects of the disclosure and, together with a general description of the disclosure given above, and the detailed description given below, serve to explain the principles of the disclosure, wherein:

FIG. 1 is a side view of a circular stapling instrument according to aspects of the disclosure;

FIG. 2 is a side view of an anvil assembly and a distal portion of an adapter assembly of the circular stapling instrument shown in FIG. 1, including an exemplary loading unit and a trocar member extending through the loading unit, prior to securing the anvil assembly to the trocar member;

FIG. 3 is a schematic view of a trans-oral introduction of the anvil assembly shown in FIG. 2, and the trans-abdominal introduction of the distal portion of the adapter assembly shown in FIG. 2, into a body cavity of a patient;

FIG. 4 is a schematic view a surgical stapling procedure using the anvil assembly and distal portion of the adapter assembly shown in FIG. 2 each secured to a section of a tubular organ and prior to attaching the anvil assembly to the trocar member; and

FIG. 5 is a schematic view of the surgical stapling procedure shown in FIG. 4 subsequent to attaching the anvil assembly to the trocar member.

### DETAILED DESCRIPTION

Aspects of the disclosure are described in detail with reference to the drawings, in which like reference numerals designate identical or corresponding elements in each of the several views. As used herein, the term “distal” refers to that portion of the adapter assembly or surgical device, or component thereof, farther from the user, while the term “proximal” refers to that portion of the adapter assembly or surgical device, or component thereof, closer to the user. As used herein, the term clinician refers to anyone involved in a surgical procedure, including but not limited to, surgeons, support staff, and other medical personnel.

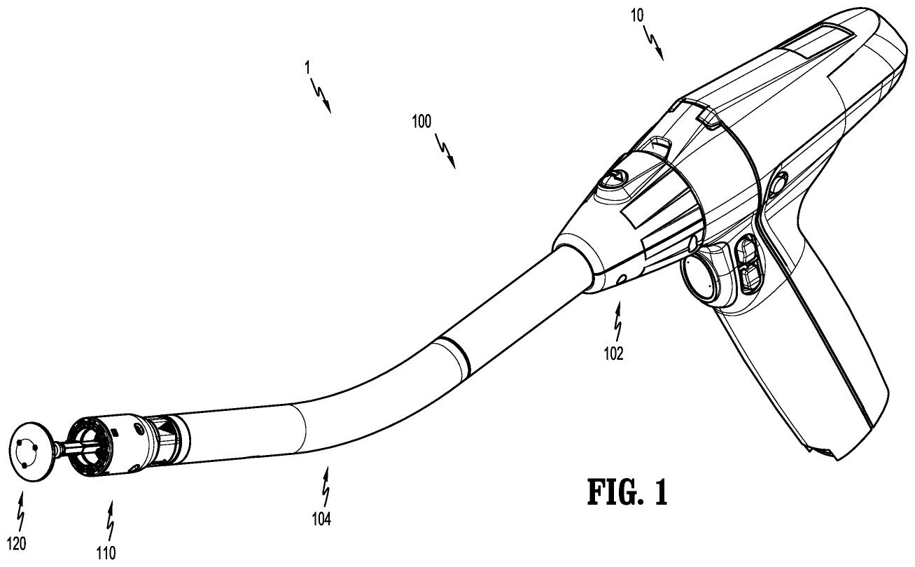

FIG. 1 illustrates a circular stapling instrument 1 according to aspects of the disclosure and will be described in detail with respect to a powered handle assembly 10. Although shown and described as relates to the powered handle assembly 10, it is envisioned that the aspects of the disclosure may be modified for use with powered and non-powered handle assemblies having various configurations. For a detailed description of an exemplary electromechanical powered handle assembly, please refer to U.S. Pat. Nos. 9,055,943 and 9,918,713, the contents of which are incorporated by reference herein in their entirety.

The circular stapling instrument 1 includes an adapter assembly 100 extending from the powered handle assembly 10. The adapter assembly 100 will only be described to the extent necessary to fully disclose the aspects of the disclosure. For a detailed description of exemplary adapter assemblies, please refer to U.S. Pat. No. 10,371,238 and U.S. Pat. App. Pub. No. 2017/0128123 (“the '123 application”), the contents of each of which are incorporated by reference herein in their entirety.

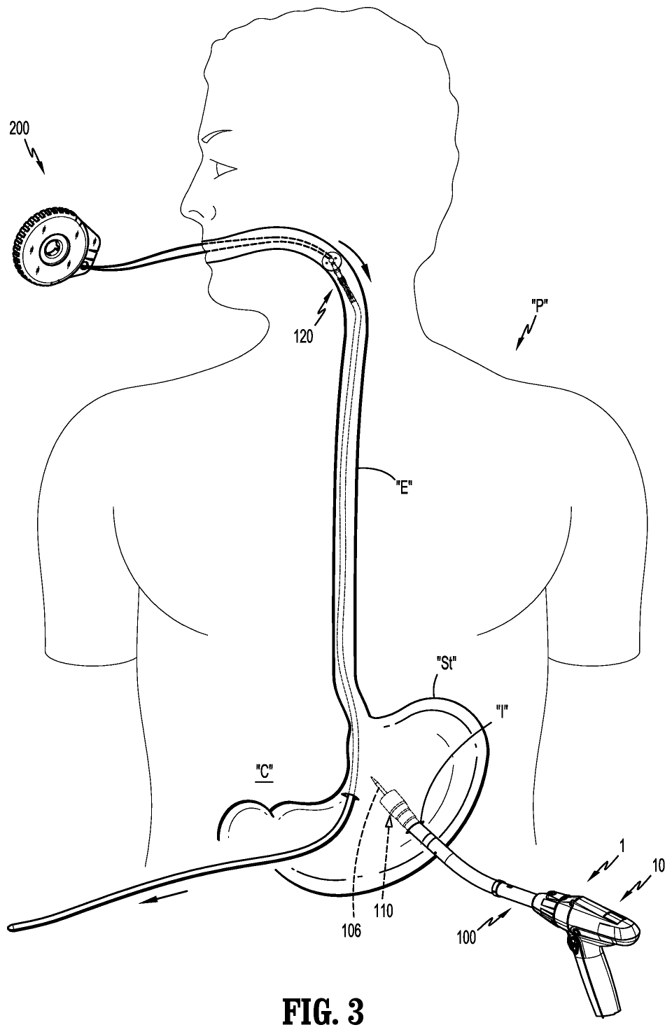

The adapter assembly 100 releasably connects to the powered handle assembly 10. The adapter assembly 100 includes a housing 102 operably connectable to the powered handle assembly 10, and an elongate body 104 extending from the housing 102. A loading unit 110 may be integrated with the adapter assembly 100, or may be releasably coupled to the adapter assembly 100 to permit reuse of the adapter assembly 100. The elongate body 104 of the adapter assembly 100 may be flexible to facilitate insertion of the loading unit 110 within a body cavity “C” (FIG. 3) of a patient “P”.

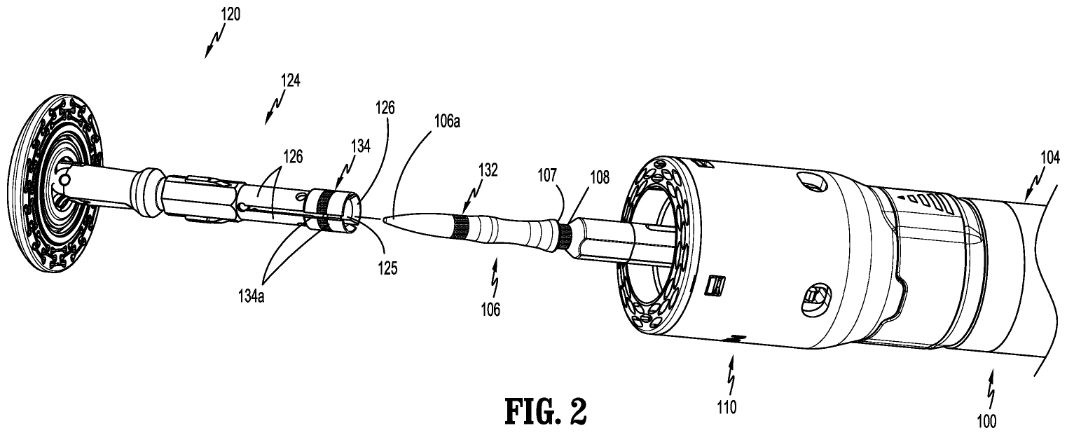

FIG. 2 illustrates a trocar member 106 extending from a distal portion of the elongate body 104 of the adapter assembly 100. The trocar member 106 is configured for releasably engaging an anvil assembly, e.g., anvil assembly 120. The trocar member 106 and anvil assembly 120 will only be described to the extent necessary to fully disclose the aspects of the disclosure. For a detail description of an exemplary trocar member, please refer the '123 application, the content of which was previously incorporated by reference herein, and for a detail description of an exemplary anvil assembly, please refer to U.S. Pat. No. 8,328,063.

The trocar member 106 extends through the loading unit 110 supported on the distal portion of the elongate body 104. The trocar member 106 includes a piercing distal tip 106a and defines a notch 107 longitudinally spaced from the distal tip 106a configured for operable engagement with legs 126 of a center rod 124 of the anvil assembly 120. In certain aspects of the disclosure, and as shown, a band 108 is disposed within or in close proximity to the notch 107. As will be described in further detail below, when the anvil assembly 120 is fully engaged with the trocar member 106, the band 108 on the trocar member 106 is covered by the legs 126 of the center rod 124 of the anvil assembly 120 and is no longer visible to the clinician. In some aspects of the disclosure, the band 108 is orange.

As described in the '123 application, the trocar member 106 is retractable within the elongate body 104 to approximate the anvil assembly 120 relative to the loading unit 110.

The trocar member 106 of the circular stapling instrument 1 (FIG. 1) includes a first magnetic member 132 positioned between the piercing distal tip 106a of the trocar member 106 and the notch 107 of the trocar member 106. As shown, the first magnetic member 132 includes a ring or sleeve. The first magnetic member 132 may be positioned on an internal or external surface of the center rod 124. It envisioned the first magnetic member 132 may include a component on each of an internal surface and an external surface of the center rod 124. In alternative aspects of the disclosure the first magnetic member 132 includes a magnetic tape or wrap that may be secured to the trocar member 106. The first magnetic member 132 may be secured to known trocar members (not shown) using adhesive, friction fit, or in any other suitable manner without modifying the trocar members. Alternatively, the trocar member 106 may including a groove for receiving the first magnetic member 132. In this manner, the first magnetic member 132 may be recessed to prevent any incidental catching of the center rod 124 of the anvil assembly 120 with the trocar member 106.

The legs 126 of the center rod 124 of the anvil assembly 120 include a second magnetic member 134. As with the first magnetic member, the second magnetic member is shown, and will be described as including a ring or sleeve. It is envisioned that the second magnetic member 134 may include a magnetic tape or wrap. The second magnetic member 134 is positioned distal of the proximal end of the center rod 124 and in such a manner that the trocar member 106, including the first magnetic member 132, is magnetically drawn towards an opening 125 formed at the distal end of the legs 126 of the center rod 124 of the anvil assembly 120. In this manner, the first and second magnetic members 132, 134 form an alignment mechanism configured to facilitate attachment of the anvil assembly 120 to the trocar member 106.

Although described as a ring, as shown, the second magnetic member 134 includes three ring sections 134a corresponding to the three legs 126 of the center rod 124 of the anvil assembly 120. In this manner, the configuration of the second magnetic member 134 accommodates flexing of the legs 126 of the center rod 124 to permit securing of the anvil assembly 120 with the trocar member 106. It is envisioned that the second magnetic member 134 may be secured to the legs 126 of the center rod 124 using adhesive, or in any other suitable manner. The second magnetic member 134 may be received on the outer surface of the legs 126 of the center rod 124, or on an inner surface of the legs 126 of the center rod 124. As with the first magnetic member 132, the second magnetic member 134 may be received within a groove such that the second magnetic member 134 is recessed to prevent catching of the center rod 124 of the anvil assembly 120 with the trocar member 106.

It is envisioned that that magnetic attraction between the first and second magnetic members 132, 134 is such that the anvil assembly 120 and the trocar member 106 facilitate alignment of the anvil assembly 120 with the trocar member 106, but no so much that the legs 126 of the center rod 124 of the anvil assembly 120, including the second magnetic member 134, cannot be passed over the first magnetic member 132 and secured to the trocar member 106 as described below.

FIG. 3 illustrates the trans-oral introduction and placement of the anvil assembly 120 of the circular stapling instrument 1 (FIG. 1) through the esophagus “E” and into the stomach “St” of a patient “P” facilitated by an anvil delivery system 200. Similarly, a distal portion of the adapter assembly 100, including the loading unit 110 and the trocar assembly 106, received in the body cavity “C” of the patient “P” through an incision (not shown) and is introduced into the stomach “St” through an incision “I” in the stomach “St”.

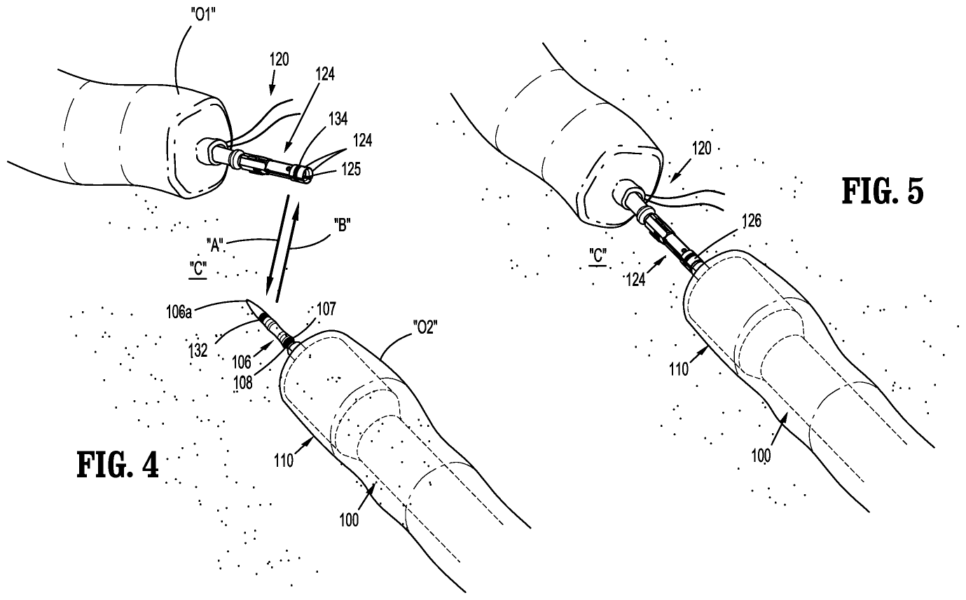

FIG. 4 illustrates the attraction between the second magnetic member 134 on the legs 126 of the center rod 124 of the anvil assembly 120 and the first magnetic member 132 on the trocar member 106, shown as arrows “A” and “B”. As shown, a first section of tubular organ “01” is secured to the anvil assembly 120 and a second section of tubular organ “02” is secured to the distal portion of the adapter assembly 100. In order to secure the first section of tubular organ “01” with the second section of tubular organ “02”, the anvil assembly 120 first secured to the trocar member 106. The attraction between the first and second magnetic members 132, 134 facilitates alignment of the distal tip 106a of the trocar member 106 with the opening 125 between the legs 126 of the center rod 124 of the anvil assembly 120.

FIG. 5 illustrates the legs 126 of the center rod 124 of the anvil assembly 120 fully engaged with the trocar member 106. More particularly, distal ends of the legs 126 are received within the notch 107 (FIG. 4) in the trocar member 106 and the band 108 disposed within the notch 107 is no longer visible. The disappearance of the band 108 is a visual indicator to the clinician that the anvil assembly 120 is secured to the trocar member 106. As noted above, it is envisioned that the magnetic attraction between the first and second magnetic members 132, 134 is such that the legs 126 of the center rod 124 of the anvil assembly 120, including the second magnetic members 134, are not prevented from sliding over and past the first magnetic member 132 on the trocar member 106. Similarly, the magnetic attraction between the first and second magnetic members 132, 134 is not so great as to prevent separation of the anvil assembly 120 from the trocar member 106 subsequent to a stapling procedure.

Persons skilled in the art will understand that the devices and methods specifically described herein and illustrated in the accompanying drawings are non-limiting exemplary aspects of the disclosure. It is envisioned that the elements and features illustrated or described in connection with one exemplary aspect may be combined with the elements and features of another without departing from the scope of the disclosure. As well, one skilled in the art will appreciate further features and advantages of the disclosure based on the above-described aspects. Accordingly, the disclosure is not to be limited by what has been particularly shown and described, except as indicated by the appended claims.

What is claimed is:

1. An adapter assembly, comprising:
an elongate body including a distal portion;
a loading unit disposed on the distal portion of the elongate body, the loading unit including a staple cartridge supporting a plurality of surgical staples;
a trocar member extending from the elongate body and through the loading unit, the trocar member including a first magnetic member supported along a length of the trocar member; and
an anvil assembly including a center rod having a plurality of legs defining an opening, the center rod including a second magnetic member supported on the plurality of legs of the center rod, the second magnetic member being magnetically attracted to the first magnetic member to direct the trocar member towards the anvil assembly.

2. The adapter assembly of claim 1, wherein the trocar member includes a piercing distal tip and a notch, the first magnetic member being disposed between the piercing distal tip and the notch.

3. The adapter assembly of claim 2, wherein the trocar member includes a visible band disposed within the notch.

4. The adapter assembly of claim 1, wherein the center rod of the anvil assembly is releasably engageable with the trocar member.

5. The adapter assembly of claim 4, wherein the trocar member includes a visible band supported along a length of the trocar member.

6. The adapter assembly of claim 5, wherein the visible band is covered by the plurality of legs of the center rod when the center rod is attached to the trocar member.

7. The adapter assembly of claim 5, wherein the visible band is orange.

8. The adapter assembly of claim 1, wherein a distal tip of the trocar member is configured to pierce tissue.

9. The adapter assembly of claim 1, wherein the first magnetic member includes a magnetic ring.

10. The adapter assembly of claim 1, wherein the second magnetic member includes a magnetic sleeve.

11. The adapter assembly of claim 1, wherein the elongate body is a component of an adapter assembly.

12. An anvil assembly for a circular stapling instrument have a trocar with a first magnetic member, the anvil assembly comprising:
a center rod having a plurality of legs defining an opening, the center rod including a second magnetic member supported on the plurality of legs of the center rod, the second magnetic member being magnetically attracted to the first magnetic member to direct the trocar member towards the anvil assembly.

13. The anvil assembly of claim 12, wherein the center rod is configured to be releasably engageable with the trocar member.

14. The anvil assembly of claim 12, wherein the second magnetic member includes a magnetic sleeve.

15. A circular stapling instrument, comprising:
a handle assembly;
an adapter assembly secured to the handle assembly, the adapter assembly including,
an elongate body including a distal portion;
a loading unit disposed on the distal portion of the elongate body, the loading unit including a staple cartridge supporting a plurality of surgical staples; and
a trocar member extending from the elongate body and through the loading unit, the trocar member including a first magnetic member supported along a length of the trocar member; and
an anvil assembly including a center rod having a plurality of legs defining an opening, the center rod including a second magnetic member supported on the plurality of legs of the center rod, the second magnetic member being magnetically attracted to the first magnetic member to direct the trocar member towards the anvil assembly.

16. The circular stapling instrument of claim 15, wherein the trocar member includes a piercing distal tip and a notch, the first magnetic member being disposed between the piercing distal tip and the notch.

17. The circular stapling instrument of claim 16, wherein the trocar member includes a visible band disposed within the notch.

18. The circular stapling instrument of claim 16, wherein the center rod of the anvil assembly is releasably engageable with the trocar member.

19. The circular stapling instrument of claim 15, wherein the trocar member includes a visible band supported along a length of the trocar member.

20. The circular stapling instrument of claim 19, wherein the visible band is covered by the plurality of legs of the center rod when the center rod is attached to the trocar member.
